(19) United States
(12) Patent Application Publication
(10) Pub. No.: US 2026/0225929 A1
(43) Pub. Date: Aug. 6, 2026

(54) ANAEROBIC DIGESTER AND METHOD OF USE THEREOF

(71) Applicant: MYGUG LIMITED, County Cork (IE)

(72) Inventor: Kieran COFFEY, County Cork (IE)

(73) Assignee: MYGUG LIMITED, County Cork (IE)

(21) Appl. No.: 19/159,167

(22) PCT Filed: Feb. 14, 2024
(86) PCT No.: PCT/EP2024/053709

§ 371 Date: Aug. 22, 2025

(30) Foreign Application Priority Data
Feb. 23, 2023 (EP) 23158221.4

(51) Int. Cl. C02F 3/00; C02F 3/28

(52) U.S. Cl. CPC C02F 3/006; C02F 3/282; C02F 3/2866; C02F 2201/005; C02F 2203/006; C02F 2209/02; C02F 2209/03; C02F 2209/38; C02F 2209/42; C02F 2209/44; C02F 2301/066

(57) ABSTRACT

An anaerobic digester apparatus has a reactor having a tank and an agitator, a feedstock inlet, a liquid outlet at an intermediate vertical level of the tank, a valved gas conduit at an upper vertical level of the tank, and a pressure sensor arranged to sense upper tank gas pressure. The controller deactivates the agitator to start an initial settlement period, upon expiry of the initial settlement period, closes the gas conduit to allow gas pressure build up in the top of the tank during a final settlement period, during said final settlement period monitors sensed gas pressure and automatically measures liquid level as said level drops, opens the gas conduit and actuates the agitator when it is determined that the liquid level has dropped to a threshold, and returns to deactivate the agitator after a pre-set agitation time has elapsed.

### INTRODUCTION

The present invention relates to anaerobic digesters.

An example of a known anaerobic digester (“AD”) is described in U.S. Pat. No. 5,185,079 (Dague). This describes an anaerobic sequence batch process carried out in a single vessel which is operated on a fill-and-draw basis in a sequential manner. When waste is entering the reactor, the vessel is mixed by biogas or liquid recirculation. Waste feeding continues until the reactor is filled to its predetermined full liquid level. The anaerobic reaction proceeds with intermittent or continuous mixing. Mixing is then discontinued, allowing the biomass to settle under quiescent conditions resulting in the formation of a low suspended solids supernatant. After sufficient settling time, supernatant is withdrawn from the reactor, thereby lowering the reactor contents to the predetermined lower liquid level. Gas or liquid recirculation mixing is then begun again along with waste feeding. Excess biomass is wasted from the reactor on a periodic basis.

The period for which organic material remains inside the anaerobic digester (“AD”) is called retention or residence time. It is divided into two types, solids retention time (“SRT”) and hydraulic retention time (“HRT”). The time associated with microorganisms present in the digester denotes SRT.

In many conventional batch digesters, the fluids, solids, and microbes are not well mixed but synchronously discharged, that is HRT=SRT. This often leads to incomplete digestion of organic substances and low digestion efficiency, which can be overcome to some extent in continuous stirred-tank reactors (CSTRs). CSTR digesters have continuous mixing of effluent and biomass with the help of agitators. The stirrers ensure sufficient mixing of materials and microbes. Much higher digestion efficiency can be achieved due to the mixing. CSTR is often operated in a continuous mode, with effluent discharged under a dynamic balance state. Advantages include the uniform distribution of materials, full contact of materials and microbes, no clogging or by-passing flow, and easy establishment of automatic control systems. However, it also has several disadvantages such as a significant loss of microbes which requires SRT as long as 30-60 days and high energy consumption for mixing.

The present invention is directed towards providing an AD which is of simpler construction than those of the art, particularly with a view to it being suitable for domestic or small business use.

### SUMMARY OF THE INVENTION

We describe an anaerobic digester apparatus comprising a reactor having a tank and an agitator, a feedstock inlet, a liquid outlet at an intermediate vertical level of the tank, a valved gas conduit at an upper level of the tank, a pressure sensor arranged to sense upper tank gas pressure, and a controller, wherein the controller is configured to:
(a) deactivate the agitator to start an initial settlement period,
(b) upon expiry of the initial settlement period, close the gas conduit to allow gas pressure build up in the top of the tank during a final settlement period,
(c) during said final settlement period monitor sensed gas pressure in the top of the tank, and to automatically measure liquid level in the tank as said level drops,
(d) open the gas conduit and actuate the agitator when it is determined that the liquid level has dropped to a threshold, and to
(e) return to step (a) after a pre-set agitation time has elapsed.

In some preferred examples, the controller is configured with a pre-set duration for the initial settlement period according to nature of the feedstock material. In some preferred examples, the apparatus comprises a supply of gas linked with the tank, and the controller is configured to inject gas to the top of the tank from said supply in order to reduce duration of the final settlement period. In some preferred examples, the controller is configured to allow manual over-ride to actuate the agitator and open the gas conduit.

In some preferred examples, the controller is configured to store a level reduction ΔL2 as triggering the end of the final settlement phase. In some preferred examples, the level reduction ΔL2 is that which brings the liquid level L down to close to the level of an inlet of the liquid outflow conduit.

In some preferred examples, the controller is configured to determine a time for start of the final settlement period as being when a blanket level B is below an inlet opening of the liquid outlet whereby primarily clear liquid L flows out through the liquid outlet under gas pressure during the final settlement period. In some preferred examples, the pressure sensor is within the gas conduit, between a valve and the tank.

In some preferred examples, the apparatus further comprises a sludge conduit below the liquid outlet (11), and the controller is configured to close the liquid conduit to allow outflow of sludge.

In some preferred examples, the apparatus further comprises a floc outlet conduit, and the controller is configured to close said liquid conduit to allow outflow of floc. In some preferred examples, the tank comprises an insulation jacket comprising a phase change material. In some preferred examples, the apparatus comprises a heater in a jacket of the tank, and the controller is configured to actuate the heater during low supply cost times such as at night.

In some preferred examples, the apparatus further comprises a balance tank for delivering digestate to the reactor tank, and a level sensor for the balance tank, in which the level sensor comprises a conduit having a lower open end at a level for immersion in biomass, and a closed upper end at a higher level, and a pressure sensor, and in which the controller is configured to determine level according to pressure sensed in said sensor conduit.

We also describe a method of operation of an anaerobic digester apparatus of any example, the method comprising steps implemented by the controller of:
deactivating the agitator to start an initial settlement period,
upon expiry of the initial settlement period, close the gas conduit to allow gas pressure build up in the top of the tank during a final settlement period,
during said final settlement period monitor sensed gas pressure and automatically estimate liquid level as said level drops,
open the gas conduit and actuate the agitator when it is determined that the liquid level has dropped to a threshold, and
return to the first step of deactivating the agitator after a pre-set agitation time has elapsed.

In some preferred examples, the controller is configured with a pre-set duration for the initial settlement period according to nature of the feedstock material. In some preferred examples, the apparatus comprises a supply of gas linked with the tank, and the controller injects gas to the top of the reactor from said supply in order to reduce duration of the final settlement period. In some preferred examples, the controller allows manual over-ride to actuate the agitator and open the gas conduit.

In some preferred examples, the controller stores a level reduction as triggering the end of the final settlement phase according to volume of outflow of liquid.

In some preferred examples, the apparatus further comprises a sludge conduit below the liquid outlet, and the controller closes the liquid conduit to allow outflow of sludge.

In some preferred examples, the apparatus further comprises a floc outlet conduit, and the controller is configured to close said liquid conduit to allow outflow of floc.

### Additional Statements

We describe an anaerobic digester apparatus comprising a reactor having a tank and an agitator, a feedstock inlet, a liquid outlet at an intermediate vertical level of the tank, a valved gas conduit at an upper vertical level of the tank, a pressure sensor arranged to sense upper tank gas pressure, and a controller, wherein the controller is configured to:
(a) deactivate the agitator to start an initial settlement period, and to
(b) upon expiry of the initial settlement period, close the gas conduit to allow gas pressure build up in the top of the tank during a final settlement period, and to,
(c) during said final settlement period monitor gas pressure and to automatically estimate or measure liquid level as said level drops, and to
(d) open the gas conduit and actuate the agitator when it is determined that the liquid level has dropped to a threshold, and to
(e) return to step (a) after a pre-set agitation time has elapsed.

In some preferred examples, the controller is configured with a pre-set duration for the initial settlement period according to nature of the feedstock material.

In some preferred examples, the apparatus comprises a supply of gas linked with the tank, and the controller is configured to inject gas to the top of the reactor from said supply in order to reduce duration of the final settlement period.

In some preferred examples, the controller is configured to allow manual over-ride to actuate the agitator and open the gas conduit.

In some preferred examples, the controller is configured to store a level reduction as triggering the end of the final settlement phase according to volume of outflow of liquid.

In some preferred examples, the apparatus further comprises a sludge conduit below the liquid outlet, and the controller is configured to close the liquid conduit to allow outflow of sludge.

In some preferred examples, the apparatus further comprises a floc outlet conduit (12), and the controller is configured to close said liquid conduit to allow outflow of floc.

We also describe a method of operation of an anaerobic digester apparatus of any example described herein, the method comprising steps implemented by the controller of:
deactivating the agitator to start an initial settlement period,
upon expiry of the initial settlement period, close the gas conduit to allow gas pressure build up in the top of the tank during a final settlement period,
during said final settlement period monitor sensed gas pressure and automatically estimate or measure liquid level as said level drops,
open the gas conduit and actuate the agitator when it is determined that the liquid level has dropped to a threshold, and
return to the step of deactivating the agitator after a pre-set agitation time has elapsed.

In some preferred examples, the controller is configured with a pre-set duration for the initial settlement period according to nature of the feedstock material.

In some preferred examples, the apparatus comprises a supply of gas linked with the reactor, and the controller injects gas to the top of the reactor from said supply in order to reduce duration of the final settlement period.

In some preferred examples, the controller allows manual over-ride to actuate the agitator and open the gas conduit.

In some preferred examples, the controller stores a level reduction as triggering the end of the final settlement phase according to volume of outflow of liquid.

In some preferred examples, the apparatus further comprises a sludge conduit below the liquid outlet, and the controller closes the liquid conduit to allow outflow of sludge.

In some preferred examples, the apparatus further comprises a floc outlet conduit, and the controller is configured to close said liquid conduit to allow outflow of floc.

### DETAILED DESCRIPTION OF THE INVENTION

The invention will be more clearly understood from the following description of some embodiments thereof, given by way of example only with reference to the accompanying drawings in which:

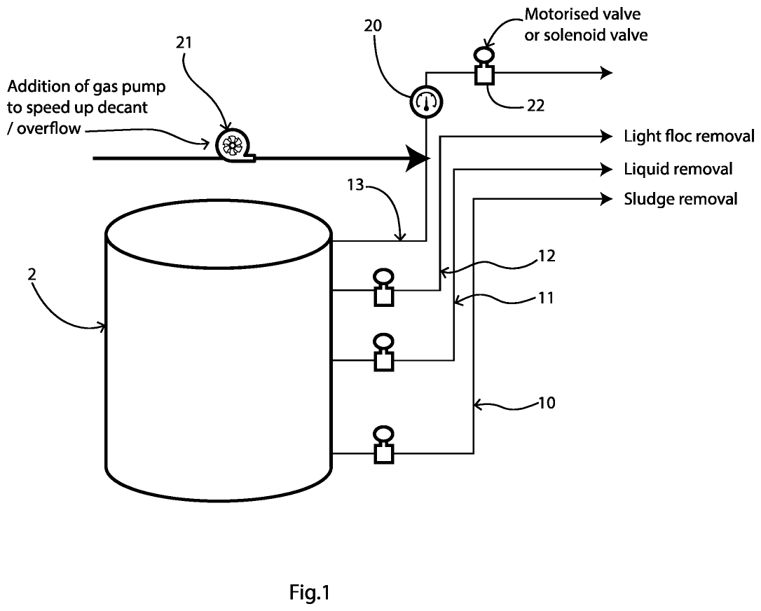

FIG. 1 diagrammatic illustration of an AD apparatus of the invention,

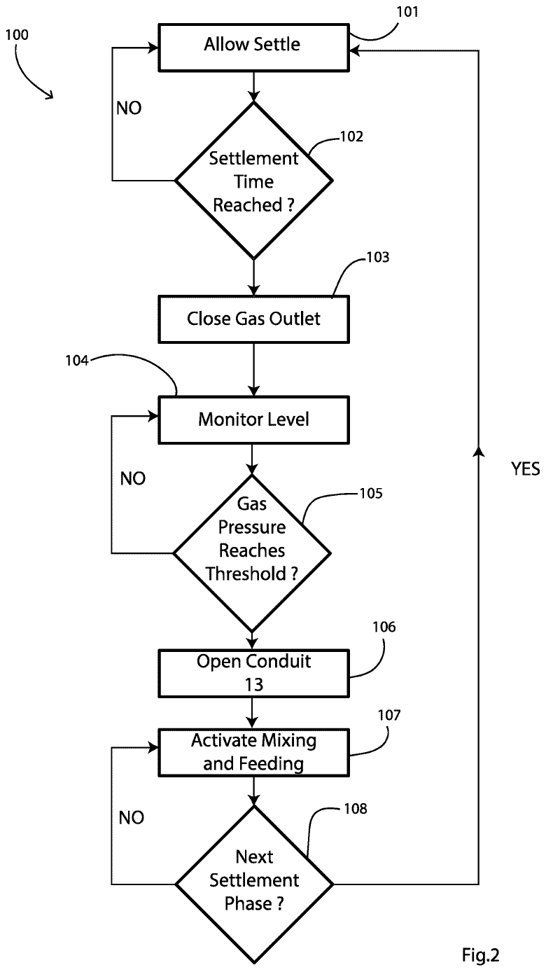

FIG. 2 is a flow diagram illustrating major control steps implemented by a controller in response to sensor inputs,

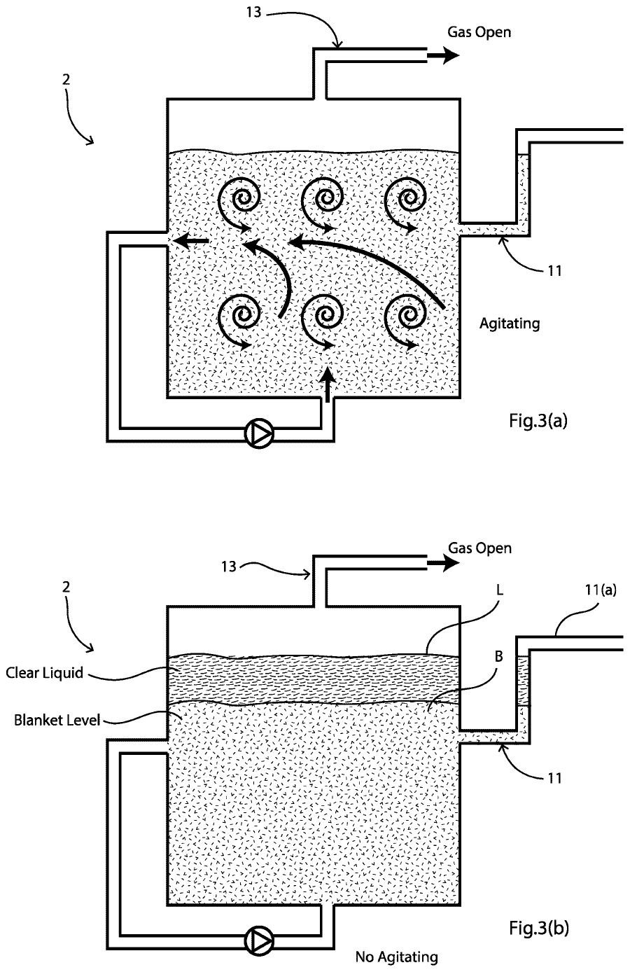

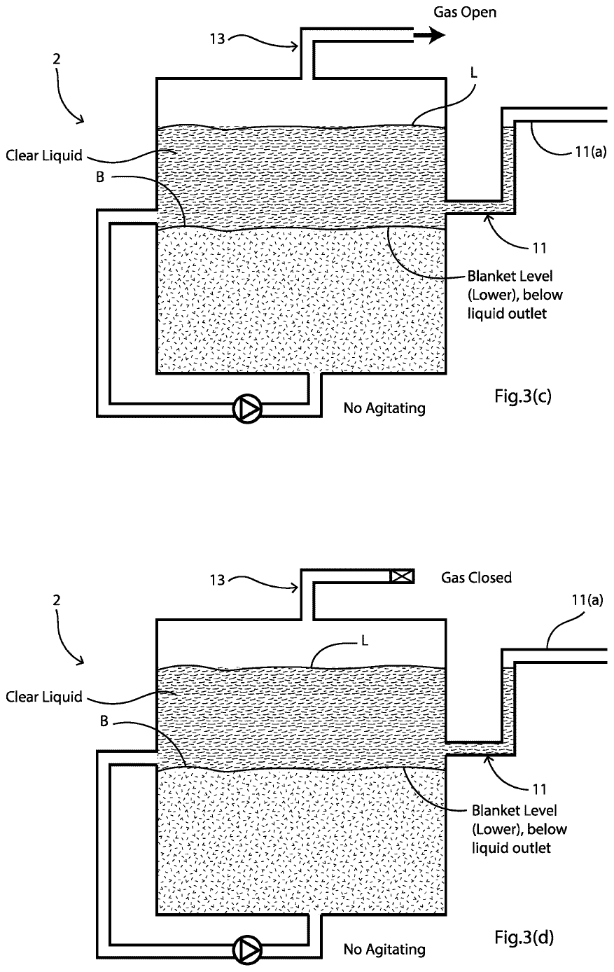

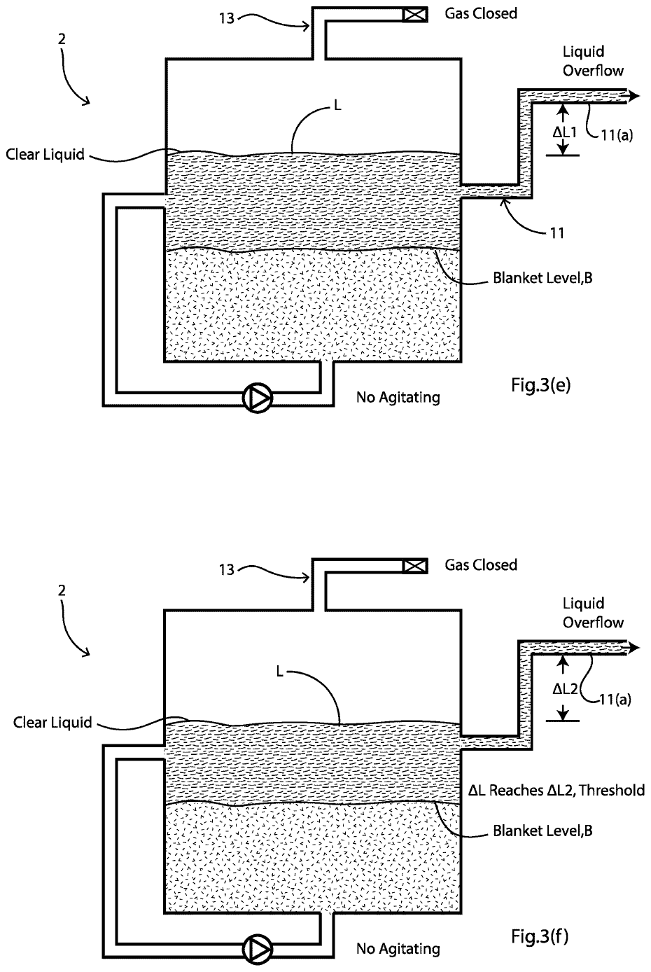

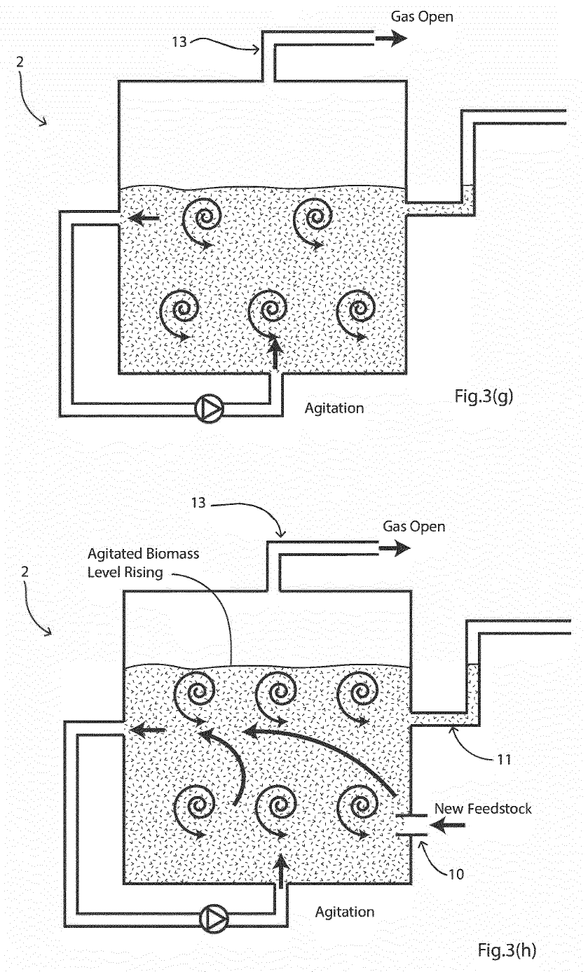

FIGS. 3(a) to (h) inclusive are cross-sectional and flow diagrams showing the major components of the reactor at different stages of operation,

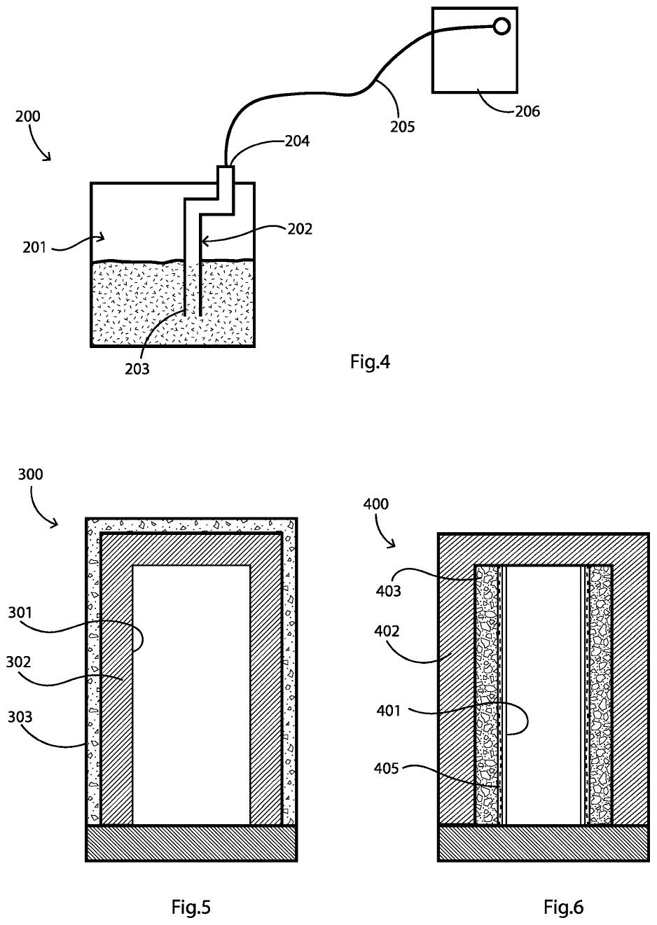

FIG. 4 is a diagram showing a balance tank which feeds biomass to the reactor and how level is sensed in the balance tank, and

FIGS. 5 and 6 are cross-sectional diagrams showing alternative reactors with features for improved temperature stability and improved energy efficiency.

### DESCRIPTION of the Embodiments

Referring to FIG. 1 an AD apparatus 1 comprises a reactor 2 comprising a cylindrical tank with an agitator (not shown, vertical axis, in lower part of the reactor 2) of conventional construction to which are connected a feedstock inlet (not shown) and three primary outlets:
a sludge removal outlet 10 with a motorized valve,
a liquid removal outlet 11 with a motorized valve,
a light floc (or “supernatant”) removal outlet 12, also with a motorized valve.

At the upper end of the reactor 2 there is a gas conduit 13 including a pressure sensor 20 and a motorized valve 22 for controlling flow of gas from the top of the reactor through this conduit.

There is an optional gas pump 21 to increase gas pressure at the top of the reactor to reduce a settlement time, if preferred. The pressure sensor 20 is open to the top of the reactor 2 and so directly measures pressure of the gas there.

The invention allows for the easy conversion of a CSTR reactor to a sequencing type system. In aerobic wastewater treatment plants this is known as an SBR or Sequencing Batch Reactor. In the case of anaerobic digestion, it could be termed as a solids concentrator, or a bacterial concentrator. The reactor tank, infeed port, and agitator components are used. The solids conduit 10, the liquid overflow conduit 11, the floc conduit 12, and the arrangement of the gas conduit 13 are the major components which would be added to a conventional reactor to provide a system of the invention at a physical component level.

At a determined time, all mixing in the reactor 2 is turned off to commence an initial settlement period. Stratification of the bacteria starts whereby solids settle and lighter material rises, leaving a volume in the reactor that has less bacteria present. The initial settlement period pertains to the speed of settlement of the anaerobic bacteria/sludge, defined at the top by a blanket level (B in the drawings). During the initial settlement period the blanket level B drops to a desired level, preferably below the inlet to the conduit 11. This could be determined according to a pre-determined settlement rate, e.g. 1 m/hr. For example, if a 500 mm drop in level is required, the settlement period at a rate of 1 m/hr will be 30 minutes. The settlement rate can be either estimated using standard settlement tests or using a typical settlement rate.

After this initial settlement has happened the valve 22 on the gas outlet conduit 13 is closed by the controller to prevent the escape of gas, and this initiates a final settlement period. As the gas continues to be produced in the digester this creates a back pressure on the liquid surface. This in turn creates an overflow in the liquid outlet 11, and the overflow is preferentially of lighter consistency, thereby concentrating the biomass with higher bacterial concentration to remain in the tank. The pressure sensor 20 on the gas line 13 provides a measurement of the drop in level in the reactor according to the algorithm: P=ρ g h, in which, ρ is density of the liquid, g is the gravitational constant, and h is the liquid level.

After this drop in level, the valve 22 on the gas line 13 is opened again and mixing can re-start. The pressure times the area of the tank can be used to determine the volume of liquid discharged from the tank. Through this simple addition of the valve 22 and the pressure sensor in the conduit 13 together with suitable programming of the controller, the apparatus 1 achieves the benefit of mixing and also of good SRT. It is proposed that this process could happen a number of times a day or possibly one time in a number of days.

Other pressure measuring instruments could be used instead of, or in addition to, a pressure sensor in the outlet conduit 13, for example a pressure bulb sensor, an ultrasonic sensor, a magnetic sensor, or and/or a capacitive liquid level sensor.

Referring to FIG. 2 a method 100 for control of the apparatus 1 is illustrated with reference also to FIGS. 3(a) to 3(h). The controller controls the components in a step 101 to provide agitation (FIG. 3(a)) and mixing of the contents with the top open to atmospheric pressure, followed by stopping the agitation to allow the reactor 2 contents to settle in the initial settlement period (FIG. 3(b)). This provides a liquid level L and a lower blanket level B, both of which also appear in the liquid outlet conduit 11 which has a portion which extends vertically alongside the tank. FIG. 3(c) shows further digestion resulting in the blanket level B being below the conduit 11 inlet, so that there is only clear liquid in the conduit 11.

As indicated by the decision step 102 and FIG. 3(d) the initial settlement time is monitored and when the blanket level B drops below the liquid conduit 11 inlet the gas outlet conduit 13 is closed by the motorised valve 22 in step 103, to initiate the final settlement period. This time period is determined by testing of the contents of the reactor to determine optimal settling times. This can vary depending on the shape of the digester, temperature within the vessel, the type of stirring, and the type of feedstock.

With the gas conduit being closed and being within the final settlement period, the pressure as detected by the gauge 20 is monitored by the controller, which executes an algorithm to provide a measurement of the liquid level L, in step 104. This algorithm is as set out above. As shown in FIG. 3(e), because the gas outlet 13 is closed the pressure above the liquid builds up, pushing the liquid level down and causing overflow via the conduit 11. This causes a difference ΔL1 between the top (horizontal) part 11(a) of the conduit 11 and the liquid level L in the tank. The pressure sensed in the conduit 13 provides a measurement of the value of ΔL1 based on the algorithm P=ρ g h.

As shown in FIG. 3(f) after the level L drops further it reaches just above the inlet of the conduit 11, and this is in one example configured as a threshold level to trigger the next stage. This level is referred to as ΔL2. An example of this threshold is a drop in liquid level (ΔL2) is 30 cm, but this will depend on the amount of liquid to be expelled during each overflow cycle.

This is shown by the decision step 105, with a determination being made as to when the gas pressure (and hence the liquid level L) reaches a threshold (the liquid level L being just above the liquid conduit inlet). When this happens, the final settlement period is ended and the gas outlet 13 opens (by opening the valve 22, FIG. 3(g)) in step 106 and mixing starts again, and feeding starts again in step 107 (FIG. 3(h). Because the conduit 13 is now open again the liquid in the outlet pipe 11 will drop to reach equilibrium with the liquid level in the tank. Mixing and feeding start again as shown by step 107 and FIG. 3(h), with introduction of fresh feedstock via the conduit 10, causing the level of the digestate to rise. The conduit 10 is alternatively used for sludge removal, but in this case is used also for fresh feedstock supply. In other examples, different conduits may be used for these operations.

This continues until the next settlement phase as determined in step 108, upon which the agitator is de-activated to start a settlement phase in step 101, back to the stage illustrated in FIG. 3(b).

It will be appreciated that, initially, the mixing in the reactor is switched off to allow for the initial settlement within the digester step (101). After a set initial settlement time has passed (102, variable depending feedstock characteristics) the automated valve 22 on the gas outlet 13 closes (step 103) to initiate the final settlement period. The gas pressure sensor 20 monitors the gas pressure level in the digester. The digester level will drop over time (as biogas is produced) which causes the pressure in the gas conduit 13 to rise and which causes a liquid overflow via the conduit 11. If the gas pressure increases to a pre-set level (indicating a pre-determined level drop) the final settlement period is ended by the gas outlet conduit 13 being opened (106) and the normal mixing and feeding cycle resumes (107) with the controller actuating the agitator. The drop in level can be calibrated to allow for either a level drop or a pre-set liquid volume overflow.

The parameters for one example test are as follows:
Reactor tank volume: 10 m³.
Type of agitator: Recirculating flow.
Type of organic feedstock: food waste.
Initial gas pressure: atmospheric.
Final gas pressure: 2.94 kPa.
Change in liquid level determined according to above: 300 mm.
Time with the agitator de activated: 2 hours.

The feeding to the digester 2 can be monitored and only when the volume of feed material equals the volume expelled the overflow cycle can be delayed, thereby allowing for variable loadings.

The conduits 10 and 12 provide additional functionality for sludge and light floc removal. For example, if the liquid overflow conduit 11 opens with the sludge and light floc conduits closed there is normal liquid overflow.

With the light floc conduit 12 open and the liquid and sludge conduits closed there is light floc overflow. With the sludge conduit 10 open and the liquid overflow and light floc conduits closed there is sludge overflow.

The overflow cycle can be speeded up by pumping gas from a gas storage reservoir back into the top of the reactor 2 until the pre-set pressure is reached.

These advantageous control aspects are possible by gas pressure monitoring as described. When the gas conduit 13 is closed the biogas being produced is trapped and creates a backpressure that expels the liquid from the tank via the overflow pipe 11 that is situated in the quiescent zone. The valve can open either on a timed basis or when the liquid level drops to a pre-determined level. When the level has dropped to the set level the valve opens again and normal operation resumes.

The solids outlet 10 and a floc outlet 12 are opened and closed either on a manual basis or timed in response to the gas sensing to provide additional advantages.

### Advantages

The SRT/HRT in the reactor can be decreased as less bacteria are exiting from the digester. This is primarily because the overhead gas pressure forces liquid out which is preferentially of a lower bacteria count, leaving a higher concentration within the tank for the ongoing AD activity.

The HRT can be decreased because there is a greater concentration of bacteria active within the digester, therefore there is more biology within the reactor to more quickly digest the organic material that is fed into the digester.

A higher loading rate can be applied to the digester as there will be a higher concentration of bacteria in the reactor. The consequence of this is that the size of the reactor can be reduced or more feedstock can be fed into a digester with this adaption.

There is a reduced capital requirement as the addition of an automated valve along with a pressure sensor are the only additional physical components required, together with applicable programming of the controller. Existing CSTR reactors can be converted to an apparatus of the invention through the addition of only two external items (pressure sensor & automated valve) and an overflow point at the correct location (if required). Reduced reactor size will result in lower power consumed for heating and mixing also.

The exit points 10 and 12 for excess sludge or light floc are optional, but where present provide the additional benefit of additional process control. Additional valving would have to be added to each exit line to control from which pipe the waste exits.

During the settle cycle, the feed cycle can proceed if it does not agitate the contents of the reactor. Also, addition of extra feedstock material will speed up the settlement cycle. The settlement process can be speeded up by pumping gas from a gas reservoir back into the top of the digester. The benefit of the introduction of gas to the top of the digester is speeding up the overflow.

Rather than waiting for the gas formation to force the overflow, a gas pump such as the pump 21 may be controlled to introduce gas from a source such as a gas storage reservoir into the top of the digester compartment. This gas pressure will force the liquid out the overflow 11.

The table below outlines the settlement requirements for different biomass settlement rates based on an outlet level being 0.5m below the “liquid overflow level” (the top horizontal part of the liquid conduit 11).

| | Distance | | |
|---|---|---|---|
| | Liquid Outlet | Time taken to | |
| | Level is below | Settle below | Settlement |
| Biomas | Liquid | Liquid Outlet | Time taken |
| Settlement | overflow Level | Level (for each | with 20% |
| Rate m/hr | (meters) | settlement rate) | safety margin |
| 1 | 0.5 | 30 | 36 |
| 0.8 | 0.5 | 38 | 45 |
| 0.6 | 0.5 | 50 | 60 |
| 0.5 | 0.5 | 60 | 72 |
| 0.4 | 0.5 | 75 | 90 |
| 0.3 | 0.5 | 100 | 120 |

Therefore, taking the example of a 0.5 m/hr settlement rate and allowing for a 20% margin of safety, a settlement period of 72 minutes will be required before the gas conduit is closed and the overflow sequence is initiated.

The amount of liquid that overflows is a function of the cross-sectional area of the tank and the back pressure that is applied to the top liquid surface. The table below gives examples of the different scenarios in regard to liquid overflow and different diameter tanks. The same principle will apply to tanks of different shapes.

| | | | | | Required | | | |
|---|---|---|---|---|---|---|---|---|
| | | | | | time to | | | |
| | | | | | drop | | | |
| | | | | | 0.5 m @ | Required | | |
| | | | | Volume | 0.5 m/hr | Pressure | Flow | |
| | | | | overflow | solids | to | Rate | |
| | Cross | Height | Volume | @ 15 | settling | achieve | of | Minutes |
| | Sectional | of | of | days | velocity | 15 days | Gas | required |
| Diameter | Area of | Tank | Tank | SRT | (20% | SRT (mm | pump | to |
| of Tank | tank (m) | (M) | (m3) | (L/day) | safety) | H2O) | L/min | overflow |
| 0.73 | 0.4183265 | 2 | 0.837 | 56 | 72 mins | 133 | 10 | 5.6 |
| 1.5 | 1.76625 | 2 | 3.533 | 236 | 72 mins | 133 | 10 | 23.6 |
| 2 | 3.14 | 2 | 6.280 | 419 | 72 mins | 133 | 10 | 41.9 |

The process of forcing liquid down can aid in the reduction of light floc/biomass and encourage the growth of faster settling biomass leading to the possibility of increasing the average biomass settlement rate and reducing the settlement times while still retaining the same SRT OR increasing the overflow. On occasion, more liquid can be forced to overflow resulting in more light floc being forced out.

### Balance Tank Pressure Sensing

Referring to FIG. 4 a balance tank system 200 for delivering biomass with the correct consistency to the reactor via the inlet 10 is illustrated. The balance tank system 200 comprises a cylindrical tank 201, and a probe comprising a vertical conduit 202 having a top end 204 linked by a tube 205 to a pressure sensor in a controller 206, and an open bottom end 203. The pressure sensor is at location 206, and the pressure is transferred to the sensor via a small tube 205 from 204. The pressure sensor at 206 is connected to the controller. As the liquid level rises the pressure rise is directly transferred to the pressure sensor via the tube 204.

With micro scale anaerobic digestion, reliable and consistent feeding of organic material to the digester is required to ensure process stability. To allow for this reliable and consistent feeding it is important to have a level sensor in the feeding/balance tank 201 that is reliable and economical. Because organic material (e.g. food waste) can vary in type and consistency this poses some difficulties as light and fatty materials will rise and heavy materials will sink. This poses problems for level switches and floating switches. Pressure bulbs and flush diaphragm sensors can be used to good effect, but these sensors are relatively expensive for use in micro digesters. To prevent air being pumped into the digester all feeding stops when the level falls below 10% full. Above 80% full a high level alert is activated. The probe 202 has the benefit of not being affected by the consistency of the biomass material, it merely relies on the volume of gas, as represented by pressure as sensed by a sensor at a level above the biomass level and so it can not be contaminated.

### Efficient Temperature Stability

In another aspect, the invention provides for improved temperature stability in the anaerobic digestion process. Referring to FIG. 5 a reactor is illustrated with the inlets and outlets omitted for clarity. The reactor 300 has a tank wall 301, an insulation jacket 302, and an outer jacket 303 of phase change material.

This arrangement helps to achieve more consistency in the operating temperature for the mesophilic digester being 37° C.-38° C. with a daily temperature variation of no more than 2-3° C. The energy requirement to maintain optimal digester temperatures is termed “parasitic load”. For micro scale digesters the energy used to maintain the temperature of the digester is preferably electrical and the parasitic load will depend on the efficiency of the insulation layer and the temperature differential between the inside and outside of the digester.

The phase change material (“PCM”) 303 is provided into the insulation jacket of the digester so that it absorbs solar energy during the day and releases this energy during the night when temperatures drop, resulting in a reduced parasitic load.

The PCM 303 will absorb solar energy during the day and the PCM and turn to liquid as it heats up. During the evening and the night as, temperatures fall the PCM will turn back into a solid and release stored energy. This heat gain and heat release decreases the electrical energy required to maintain digester temperature making it more efficient.

Furthermore, a PCM may be added on the inside of the digester layer. Referring to FIG. 6 a reactor 400 has a tank wall 401, an electric element 405 immediately outside the wall 401, and outside of that the jacket has a layer 403 of PCM, and outside that again there is a layer of insulation 402.

The digester heating controls are set up to only come during times of “Night rate electricity”. The PCM 403 will absorb additional energy at night and release this energy during the day. Although this will not reduce the parasitic load it will reduce the running cost of the digester.

The PCM material may be of any suitable type using in buildings for example be a bio-based phase change material such as of the type described in “Bio-Based Phase Change Materials Incorporated in Lignocellulose Matrix for Energy Storage in Buildings—A Review” Meysam Nazari, Mohamed Jebrane, and Nasko Terziev Department of Forest Biomaterials and Technology, Swedish University of Agricultural Sciences, Vallvägen 9C, 750 07 Uppsala, Sweden; meysam.nazari@slu.se (M.N.); nasko.terziev@slu.se (N.T.), http://dx.doi.org/10.3390/en13123065.

The PCM 403 will have a phase change temperature close to that of the digester (37, 38° C.). The electrical heater will only be used when reduced electricity costs are available (e.g. nighttime or when low cost electricity is available). When the heater comes on the PCM will absorb and store some of this electrical energy. At times when the electricity is at full cost the energy the PCM has absorbed will be released, thereby maintaining the temperature within the digester.

An outer skin layer of Perspex, polycarbonate can be introduced to aid in absorbing more solar energy (used on either or both of the above arrangements).

Components of embodiments can be employed in other embodiments in a manner as would be understood by a person of ordinary skill in the art. The invention is not limited to the embodiments described but may be varied in construction and detail.

1. An anaerobic digester apparatus comprising:
a reactor having a tank and an agitator,
a feedstock inlet,
a liquid outlet at an intermediate vertical level of the tank,
a valved gas conduit at an upper level of the tank,
a pressure sensor arranged to sense upper tank gas pressure, and
a controller,
wherein the controller is configured to:
(a) deactivate the agitator to start an initial settlement period,
(b) upon expiry of the initial settlement period, close the gas conduit to allow gas pressure build up in the top of the tank during a final settlement period,
(c) during said final settlement period monitor sensed gas pressure in the top of the tank, and to automatically measure liquid level L in the tank as said level drops,
(d) open the gas conduit and actuate the agitator when it is determined that the liquid level has dropped to a threshold, and to
(e) return to step (a) after a pre-set agitation time has elapsed.

2. The apparatus as claimed in claim 1, wherein the controller is configured with a pre-set duration for the initial settlement period according to nature of the feedstock material.

3. The apparatus as claimed in claim 1, wherein the apparatus comprises a supply of gas linked with the tank, and the controller is configured to inject gas to the top of the tank from said supply in order to reduce duration of the final settlement period.

4. The apparatus as claimed in claim 1, wherein the controller is configured to allow manual over-ride to actuate the agitator and open the gas conduit.

5. The apparatus as claimed in claim 1, wherein the controller is configured to store a level reduction as triggering the end of the final settlement phase.

6. The apparatus as claimed in claim 15, wherein the controller is configured to store a level reduction ΔL2 as triggering the end of the final settlement phase; and wherein the level reduction ΔL2 is that which brings the liquid level L down to close to the level of an inlet of the liquid outflow conduit.

7. The apparatus as claimed in claim 1, wherein the controller is configured to store a level reduction ΔL2 as triggering the end of the final settlement phase; and wherein the level reduction ΔL2 is that which brings the liquid level L down to close to the level of an inlet of the liquid outflow conduit; and wherein the controller is configured to determine a time for start of the final settlement period as being when a blanket level B is below an inlet opening of the liquid outlet whereby primarily clear liquid L flows out through the liquid outlet under gas pressure during the final settlement period.

8. The apparatus as claimed in claim 1, wherein the pressure sensor is within the gas conduit, between a valve and the tank.

9. The apparatus as claimed in claim 1, wherein the apparatus further comprises a sludge conduit below the liquid outlet, and the controller is configured to close the liquid conduit to allow outflow of sludge.

10. The apparatus as claimed in claim 1, wherein the apparatus further comprises a floc outlet conduit, and the controller is configured to close said liquid conduit to allow outflow of floc.

11. The apparatus as claimed in claim 1, wherein the tank comprises an insulation jacket comprising a phase change material.

12. The apparatus as claimed in claim 1, wherein the tank comprises an insulation jacket comprising a phase change material; and wherein the apparatus comprises a heater in a jacket of the tank, and the controller is configured to actuate the heater during low supply cost times such as at night.

13. The apparatus as claimed in claim 1, further comprising a balance tank for delivering digestate to the reactor tank, and a level sensor for the balance tank, in which the level sensor comprises a conduit having a lower open end at a level for immersion in biomass, and a closed upper end at a higher level, and a pressure sensor, and in which the controller is configured to determine level according to pressure sensed in said sensor conduit.

14. A method of operation of an anaerobic digester apparatus, said apparatus comprising:
a reactor having a tank and an agitator,
a feedstock inlet,
a liquid outlet at an intermediate vertical level of the tank,
a valved gas conduit at an upper level of the tank,
a pressure sensor arranged to sense upper tank gas pressure, and
a controller,
wherein the controller is configured to:
(a) deactivate the agitator to start an initial settlement period,
(b) upon expiry of the initial settlement period, close the gas conduit to allow gas pressure build up in the top of the tank during a final settlement period,
(c) during said final settlement period monitor sensed gas pressure in the top of the tank, and to automatically measure liquid level L in the tank as said level drops,
(d) open the gas conduit and actuate the agitator when it is determined that the liquid level has dropped to a threshold, and to return to step (a) after a pre-set agitation time has elapsed;
the method comprising steps implemented by the controller of:
deactivating the agitator to start an initial settlement period,
upon expiry of the initial settlement period, close the gas conduit to allow gas pressure build up in the top of the tank during a final settlement period,
during said final settlement period monitor sensed gas pressure and automatically estimate liquid level as said level drops,
open the gas conduit and actuate the agitator when it is determined that the liquid level has dropped to a threshold, and
return to the first step of deactivating the agitator after a pre-set agitation time has elapsed.

15. The method as claimed in claim 14, wherein the controller is configured with a pre-set duration for the initial settlement period according to nature of the feedstock material.

16. The method as claimed in claim 14, wherein the apparatus comprises a supply of gas linked with the tank, and the controller injects gas to the top of the reactor from said supply in order to reduce duration of the final settlement period.

17. The method as claimed in any of claim 14, wherein the controller allows manual over-ride to actuate the agitator and open the gas conduit.

18. The method as claimed in any of claim 14, wherein the controller stores a level reduction as triggering the end of the final settlement phase according to volume of outflow of liquid.

19. The method as claimed in claim 14, wherein the apparatus further comprises a sludge conduit below the liquid outlet, and the controller closes the liquid conduit to allow outflow of sludge.

20. The method as claimed in claim 14, wherein the apparatus further comprises a floc outlet conduit, and the controller is configured to close said liquid conduit to allow outflow of floc.
